(19) United States
(12) Patent Application Publication
(10) Pub. No.: US 2020/0046712 A1
(43) Pub. Date: Feb. 13, 2020

(54) PROCESS FOR MAKING 1,3,7- TRIMETHYLURIC ACID WATER SOLUBLE SALTS OR CO-CRYSTALS AND THEIR USAGE IN DIETARY SUPPLEMENTS OR FUNCTIONAL FOODS FORMULATIONS

(71) Applicants: Arcadia Biotechnology Ltd., Shanghai (CN); American Supplement Consulting LLC, MCKinney, TX (US)

(72) Inventors: Xichen Yuan, Shanghai (CN); Dragos Miron Alevizache, MCKinney, TX (US)

(73) Assignees: Arcadia Biotechnology Ltd., Shanghai (CN); American Supplement Consulting LLC, MCKinney, TX (US)

(21) Appl. No.: 16/100,266

(22) Filed: Aug. 10, 2018

(51) Int. Cl. A61K 31/522; A61K 45/06; A61K 9/00; A61P 25/22; A23L 33/10

(52) U.S. Cl. CPC A61K 31/522; A61K 45/06; A61K 9/0053; A23V 2200/322; A61P 25/22; A23L 33/10; A23V 2002/00; A61K 9/0095

(57) ABSTRACT

The disclosure provides palatable nutritional supplements, foods cosmetic or pharmaceutical acceptable compositions that can be administrated orally, topically or parenterally in a solid, aqueous, cream, gel or ointment dosage form, containing one or more of the purine alkaloids bioactive compounds including 1,3,7-trimethyluric acid salts, for example water soluble mineral salts thereof such as 1,3,7-trimethyluric acid sodium, calcium, magnesium, potassium salts but not limited to these or salts in a form of citrates, malates, glutarates, fumarates, adipates, phosphates, nitrates, hydrochlorites, sulfates, succinates, acetates salts but not limited to these; it also provide compositions and usage of 1,3,7-trimethyluric acid, 1,7-dimethyluric acid, 1-methyluric acid, 9-methyluric acid, 3,7-dimethyluric acid in a form of salts or co-crystals that is easily soluble in water and not bitter or easily to be masked and flavored when dissolved in a natural or artificial flavored powder drink mix or a ready to drink natural or artificial flavored aqueous formulation.

### TECHNICAL FIELD

The present invention generally relates to 1,3,7-trimethyluric acid (8-oxocaffeine) salts and co-crystals, to the methods and compositions for supplementation of a diet by usage of 1,3,7-trimethyluric acid as dietary supplements and functional foods related thereto, and more particularly relates to 1,3,7-trimethyluric acid (8-oxocaffeine) salts or co-crystals.

### BACKGROUND

1,3,7-trimethyluric acid, also referred to as trimethyluric acid (8-oxycaffeine), is a purine alkaloid that is produced in some plants and occurs as a minor metabolite of caffeine in humans. It is an oxopurine in which the purine ring is substituted by oxo groups at positions 2, 6, and 8, and the nitrogens at positions 1, 3, and 7 are substituted by methyl groups.

It is also a minor metabolite of caffeine. It is abundantly present in green and roasted coffee beans of different origins, partially fermented (pouchong) and black teas.

Masking bitterness and aftertaste of drink formulations is not an easy task and requires the addition of masking or blockers bitterness chemicals components into the formulations. It brings disadvantage for formulators and could have a serious impact in overall cost.

Accordingly, it is desirable to mask the bitterness and aftertaste of drink fomulations without the current disadvantages for formulators. In addition, it is desirable to mask the bitterness and aftertaste of drink formulations without having a serious impact on overall cost. Furthermore, other desirable features and characteristics of the present invention will become apparent from the subsequent detailed description of the invention and the appended claims, taken in conjunction with the accompanying drawings and this background of the invention.

### BRIEF SUMMARY

The present disclosure relates to methods and compositions for supplementation of a diet by usage of 1,3,7-trimethyluric acid salts in the future.

This disclosure relates specifically to this compound and its salts such as sodium, calcium, magnesium, potassium salts and their hydrates forms.

In addition, this disclosure of the present disclosure relates to dietary supplements and foods in powder, tablet or capsule dosage forms, liquid or gel formulations, containing 1,3,7-trimethyluric acid salts.

In addition, this disclosure of the present disclosure relates to nutritional supplements, in powder, tablet or capsule dosage forms, as solutions or suspensions, aqueous formulations, which may be used as stimulant of the central nervous system and metabolic stimulant. It may also be used medically to increase individuals' experiences improvement of at least one of mood, energy, focus, concentration or libido enhancer, anxiolytic or fatigue reducer.

In a non-limiting embodiment, a composition for oral administration to a mammalian subject as dietary supplements or food products, wherein it comprises at least one compound selected from: a) soluble 1,3,7-trimethyluric acid salts (8-oxocaffeine soluble salts), as well as any derivatives or related substances, b) co-crystals of 1,3,7-trimethyluric acid with any other mineral, vitamin, herbs or herbal extract, amino acids or natural metabolites of body, that are formed by any co-crystallization technique, selected from slow evaporation, mechano-chemical methods, crystallization from melt, slurry crystallization, solvent free co-crystallization, supercritical fluid, twin screw extrusion, sono-chemical methods and such, 1,3,7-trimethyluric, c) 1,7-dimethyluric acid, 1-methyluric acid, 3,7-dimethyluric acid, 9-methyluric acid salts, d) co-crystals of 1,7-dimethyluric acid, 1-methyluric acid, 3,7-dimethyluric acid, 9-methyluric acid and their salts with any other mineral, vitamin, herbs or herbal extract, amino acids or natural metabolites of body, that are formed by chemical alteration or formed by any co crystallization technique selected from slow evaporation, mechano-chemical methods, crystallization from melt, slurry crystallization, solvent free co-crystallization, supercritical fluid, twin screw extrusion, sono-chemical methods and such, and e) if administrated in liquid or semi-solid forms, at least one acid regulator or a buffer having a pKa range of about 1.5-9.0 so that the composition remains stable in aqueous, topical cream, gel or ointment dosage form.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

1,3,7-trimethyluric acid (8-oxocaffeine) and its salts offer some advantages when compared to caffeine, theacrine, theobromine, theophylline, liberine or methy-lliberine with the addition of the other benefits included in the summary below:

Some of the benefits of the present disclosure include:
1,3,7-trimethyluric acid (8-oxocaffeine) salts or co-crystals increases mood, focus, concentration, libido, reduces anxiolytic and fatigue, without any side effects that last for an extended period of time after administration,
1,3,7-trimethyluric acid (8-oxocaffeine) salts or co-crystals enhances physical activity levels in an effective dose;
1,3,7-trimethyluric acid (8-oxocaffeine) salts or co-crystals in combination with other psychostimulants have a synergistic effect that enhances the benefits described above.
1,3,7-trimethyluric acid (8-oxocaffeine) sodium salts have a better solubility in water in an effective dose (e.g. 500 mg dissolved completely upon stirring in 8 oz of room temperature water) therefore have a better bioavailability;
1,3,7-trimethyluric acid (8-oxocaffeine) leave no bitterness after taste when formulated in flavored mixes or ready to drink formulations, making the usage of bitterness blockers or taste maskers as well as coating processing for the ingredient prior to be used for drinks formulations unnecessary; any coating applied to caffeine, theacrine or liberine or methyl-liberine and their related compounds can easily affect their bioavailability and also change the metabolism pathway of them.

In one aspect, the present disclosure provides a composition for oral administration to a mammalian subject as dietary supplements or food products, wherein it comprises at least one compound selected from:

a) soluble 1,3,7-trimethyluric acid salts (8-oxocaffeine soluble salts), as well as any derivatives or related substances,

b) co-crystals of 1,3,7-trimethyluric acid with any other mineral, vitamin, herbs or herbal extract, amino acids or natural metabolites of body, that are formed by any co-crystallization technique, selected from slow evaporation, mechano-chemical methods, crystallization from melt, slurry crystallization, solvent free co-crystallization, supercritical fluid, twin screw extrusion or sono-chemical methods;

c) 1,7-dimethyluric acid, 1-methyluric acid, 3,7-dimethyluric acid, 9-methyluric acid salts,

d) co-crystals of 1,7-dimethyluric acid, 1-methyluric acid, 3,7-dimethyluric acid, 9-methyluric acid and their salts with any other mineral, vitamin, herbs or herbal extract, amino acids or natural metabolites of body, that are formed by chemical alteration or formed by any co crystallization technique selected from slow evaporation, mechano-chemical methods, crystallization from melt, slurry crystallization, solvent free co-crystallization, supercritical fluid, twin screw extrusion, sono-chemical methods; and

e) if administrated in liquid or semi-solid forms, at least one acid regulator or a buffer having a pKa range of about 1.5-9.0 so that the composition remains stable in aqueous, topical cream, gel or ointment dosage form.

In an alternative embodiment, in the composition, said 1,3,7-trimethyluric acid (8-oxocaffeine) salts are present in said dosage form of solid, liquid or semi-solid, in any amount between about 0.01% and about 10% by weight based on the total weight of said composition, including all percentages and ranges of percentages there between.

In an alternative embodiment, in the composition, said 1,3,7-trimethyluric acid (8-oxocaffeine) salts or 1,7-dimethyluric acid, 1-methyluric acid, 3,7-dimethyluric acid salts are present in said dosage form of solid, liquid or semi-solid in any amount between about 0.01% and about 10% by weight based on the total weight of said composition, including all percentages and ranges of percentages there between.

In an alternative embodiment, the composition is further combined with one or more materials selected from the group consisting of: purines, xanthines or methylated xanthines, methylated forms of uric acid, synephrine, methyl synephrine, citrus aurantium, yerba mate, green tea extracts, catechols, yohimbine species, cocoa extract, coffee bean extract, chlorogenic acid, raspberry ketone, hydroxycitrate, conjugated linoleic acid, naringin, black pepper, guggul, guarana, cola nut, beta-phenylethylamine, colostrum, coleus forskohlli, rhodiola, garcinia cambogia, ashwagandha, ginseng species, gingko, scutellaria, liquorice, dehydroepiandrosterone (DHEA), pregnenolone, tyrosine, N-Acetyl Tyrosine, glucuronolactone, taurine, choline, CDP-choline, alpha glycerol phosphatidyl choline, L-carnitne and L-carnitine derivatives, 5-hydroxytryptophan, melatonin, γ-aminobutyric acid), beta hydrohybutyric acid salts (calcium, magnesium, sodium and potassium D-β-hydroxybutyric acid DL-β-hydroxybutyric acid or L-β-hydroxybutyrate), phenylethylamine, scetetium tortosum (and Mesembrrine Alkaloids), Dendrobium candidum, quinoline quinone, ubiquinone (O), nicotinamide riboside, Huperzine A (Chinese gladiolus or agaric, L-dopa, Pinus sylvestris, 2-(dimethylamino)ethanol, medium chain triglyceride, pterostilbenes, steroids, resveratrol, berberine, D-pinitol, tyramine, hordenine, agmatine, betaine, alanine, beta alanine, branch chain amino acids, gamma-butyro betaine and combination thereof in the form of synthetic or natural sources, in said dosage form of solid or aqueous composition in any amount between about 0.01% and about 10% by weight based on the total weight of said composition, including all percentages and ranges of percentages there between.

In an alternative embodiment, the composition further comprises one or more excipients selected from the group consisting of: flavoring agents, colorants, viscosity modifiers, preservatives, fragrances, amino acids and their salts, vitamins, minerals, essential fatty acids, enzymes, co-enzymes, mono-glycerides, di-glycerides, tri-glyceride ester oils emulsifiers, hydrolyzed proteins, whey protein, stabilizers, flow modifiers, viscosity improvers, chelating agents, anti-oxidants, anti-microbials benzoates or sorbates, alcohols, esters of para-hydroxybenzoic acid, propionates, preservatives, surfactants, including anionic, cationic or nonionic surfactants, or combinations thereof.

In an alternative embodiment, in the composition, the total amount of said one or more excipients is in an amount between about 0.01% and about 10% by weight based on the total weight of said composition.

In an alternative embodiment, in the composition, the dietary supplements or food products further includes beverages, including, energy drinks of any volume which may contain any dietary ingredient in any amount, wherein vitamins and minerals are dissolved, vitamins and mineral water, alkaline or buffered water, proteins and milk products, soy products, ice cream, yoghurt, fruit juices, vegetable juices, any above-mentioned components, or combinations thereof.

In an alternative embodiment, in the composition, said beverages include one or more natural components, said one or more natural components account for about 0.1% to about 99% by weight based on the total weight of said composition.

In an alternative embodiment, the composition comprises an anti-microbial preservative in an effective amount to inhibit microbial growth.

In an alternative embodiment, in the composition, said preservative comprises a preservative selected from the group consisting of: one or more esters of para-hydroxy benzoic acid, propionates, and one or more sorbates.

In an alternative embodiment, in the composition, a buffering solution chosen from phosphates, phophoric acid, acetates, carbonates or bicarbonates, citrates, but not limited thereto may be added to control and mention the desired pH.

In another aspect, the present disclosure provides a method for administering a bioavailable form of 1,3,7-trimethyluric acid to a mammalian subject wherein said method comprises the steps of: preparing the composition as mentioned above; and orally administering said composition.

In another aspect, the present disclosure provides a method for administering a bioavailable form of 1,3,7-trimethyluric acid salt to a mammalian subject wherein said method comprises the steps of: preparing the composition according to claim 10, and orally administering said composition.

In an alternative embodiment, in the above-mentioned method for administering the composition, wherein said method further comprises upon administration of the composition, the individual experiences improvement of at least one of moods, energy, focus, concentration or libido, reduction of anxiolytic or fatigue.

Stable solid and aqueous compositions of matter useful for oral administration of at least one biologically-active form of a purine alkaloid compound structurally related to caffeine to a mammalian subject, the compositions comprise:

For tablets, capsules, softgels, gels or powder mixes compositions:
a) 1,3,7-trimethyluric acid sodium salt
b) 1,3,7-trimethyluric acid and 1,3,7-trimethyluric acid sodium salt
c) 1,7-dimethyluric acid, 1-methyluric acid, 3,7-dimethyluric acid, 9-methyluric acid and their salts
d) Co-crystals of 1,3,7-trimethyluric acids obtained by any co-crystallization processes
e) Co-crystals forms of theacrine, liberine and methyl-iberine

For example, for powder mixes and aqueous compositions:
a) 1,3,7-trimethyluric acid sodium salts or co crystals
b) Purified Water
c) Carbone dioxide (for sparkling drinks only)

In one embodiment, the composition provides said 1,3,7-trimethyluric acid or 1,3,7-trimethyluric acid sodium salt are present in said aqueous composition in any amount between about 0.01% and about 10% by weight based on the total weight of said composition, including all percentages and ranges of percentages there between.

In one embodiment, the composition of the present disclosure further comprises one or more nutritional materials selected from the group consisting of: purines, xanthines or methylated xanthines, methylated forms of uric acid, synephrine, methyl synephrine, citrus aurantium, yerba mate, green tea extracts, catechols, yohimbine species, cocoa extract, coffee bean extract, chlorogenic acid, raspberry ketone, hydroxycitrate, conjugated linoleic acid, naringin, black pepper, guggul, guarana, cola nut, beta-phenylethylamine, colostrum, coleus forskohlli, rhodiola, garcinia cambogia, ashwagandha, ginseng species, gingko, scutellaria, liquorice, dehydroepiandrosterone (DHEA), pregnenolone, tyrosine, N-Acetyl Tyrosine, glucuronolactone, taurine, choline, CDP-choline, alpha glycerol phosphatidyl choline, L-carnitne and L-carnitine derivatives, 5-hydroxytryptophan, melatonin, γ-aminobutyric acid), beta hydrohybutyric acid salts (calcium, magnesium, sodium and potassium D-β-hydroxybutyric acid DL-β-hydroxybutyric acid or L-β-hydroxybutyrate), phenylethylamine, scetetium tortosum (and Mesembrrine Alkaloids), Dendrobium candidum, quinoline quinone, ubiquinone (O), nicotinamide riboside, Huperzine A (Chinese gladiolus or agaric, L-dopa, Pinus sylvestris, 2-(dimethylamino)ethanol, medium chain triglyceride, pterostilbenes, steroids, resveratrol, berberine, D-pinitol, tyramine, hordenine, agmatine, betaine, alanine, beta alanine, branch chain amino acids, gamma-butyro betaine in the form of synthetic or natural derived are present in said any solid dosage forms or aqueous composition in any amount between about 0.01% and about 10% by weight based on the total weight of said composition, including all percentages and ranges of percentages there between.

In one embodiment, in the composition of the present disclosure, the total amount of said one or more nutritional adjuvant materials present is present in any amount between about 0.01% and about 10% by weight based on the total weight of said composition.

In one embodiment, the composition of the present disclosure further comprises one or more natural beverages, including without limitation, milk products, soy products, ice cream, yoghurt, citrus fruit juices, non-citrus fruit juices, and vegetable juices, or components of any of the foregoing.

In one embodiment, the composition of the present disclosure has said one or more natural beverages is present in any amount between about 0.1% and about 99% by weight based on the total weight of said composition.

The present disclosure also includes a method for increasing physical activity in a mammalian subject, said method comprising the steps of: preparing a composition according to the present disclosure; and orally administering the composition to said mammalian subject.

The present disclosure also includes a method for increasing mood, focus, concentration, libido enhancer, anxiolytic and fatigue reducer without crash side effects that maintains it for extended time.

The present disclosure also includes a method for enhanced activity levels in a dose-dependent manner in a mammalian subject, said method comprising the steps of: preparing a composition according to the present disclosure; and orally administering the composition to said mammalian subject.

The present disclosure also contemplates a method for increasing endurance in a mammalian subject, said method comprising the steps of: preparing a composition according to the present disclosure; and orally administering the composition to said mammalian subject.

The present disclosure also contemplates a method in a mammalian subject, said method comprising the steps of: preparing a composition according to the present disclosure; and orally administering the composition to said mammalian subject wherein the administering is of one or more of purine alkaloids described herein in combination with other psychostimulants in any possible proportion in order to further enhance the benefits according to the methods of the present disclosure.

### DETAILED DESCRIPTION OF THE PREFERRED EMBODIMENTS

### Chemical Structures:

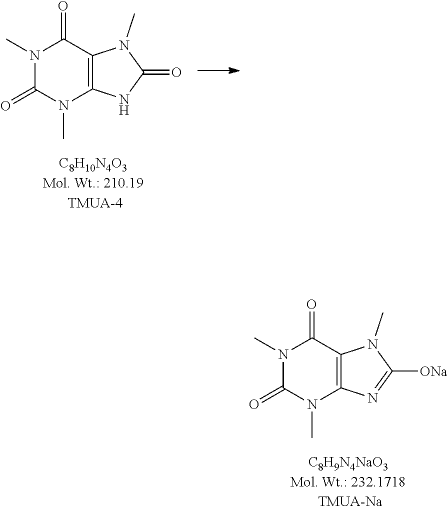

TMUA-4=trimethyluric acid
TMUA-Na=Trimethyluric acid sodium salt

### Example 1: Production of 1,3,7-Trimethyluric Acid Sodium Salt

Salts of 1,3,7-trimethyluric acid can be form by following procedure:

In a suitable reacting vessel add a stoichiometric amount of 1,3,7-trimethyluric acid; slowly add water and sodium hydroxide and stir the whole mix to dissolve; measure pH and keep pH to slightly alkaline such as pH 8, most preferable between 7 and 14. Heat can be applied if necessarily to complete dissolution; a suitable grade isopropyl alcohol is then added to the reaction mass and stirred for a period of around 20 minutes to few hours, most preferred 1 hour or until the formed salt precipitate or crystallized form solution; after the complete salt formation, the salt is filtered and dried. Similar salts of calcium, magnesium, potassium can be formed with 1,3,7-trimethyluric acid by usage of suitable salts of such as for example using hydroxides, carbonates, phosphates, peroxides, etc. It is understood that these are just examples and do not limited the disclosure to these salts.

In a preferred embodiment, the compounds or compositions can be administrated as in the following examples:

### Example 2

Drink Formula:

Sodium salt of 1,3,7-trimethyluric acid or other suitable salt of such is simply added to the water or any flavored drink; also blends can be formed by mixing sodium salt of 1,3,7-trimethyluric acid with any dietary ingredient of interest, adding the flavor profile (flavor, organic acids such as citric, malic, tartaric or a combination of these and the sweetener that can be chosen from natural or artificial kinds such as sugars, sugar alcohols such as xylitol, mannitol, trehalose, stevia, Lo han guo but not limited to those).

If preferred, the drink can be carbonated by using CO₂ gas or make it effervescent by using any carbonate/bicarbonates salts such as sodium, potassium salts.

Various liquid formulations of 1,3,7-trimethyluric sodium salt or any water-soluble salt available with dosages comprised between 0.01% and about 20% (or more) by weight based on the total weight, suitable for oral administration to mammalian subjects according to this disclosure may be caused to have any pH in the range of between about 1.5 and about 10.5 as desired, by adjusting such compositions using additions of appropriate amounts of strong or weak acids or bases, including but not limited to aqueous mineral acids including hydrochloric acid, phosphoric acid or phosphate available salts or bases including but not limited to sodium hydroxide, sodium carbonate or bicarbonate etc., as well as alkaline amino acids such as arginine base.

To prepare a composition according to one embodiment of the present disclosure, one may simply add a desired amount of 1,3,7-Trimethyluric acid salts to a selected volume of water, and sufficient stirring is affected to cause dissolution of the 1,3,7-Trimethyluric acid salts to afford an aqueous composition.

Furthermore, according to one embodiment, the solution may be buffered before the addition of 1,3,7-trimethyluric acid salts or the solution may be made more acidic or alkaline prior to the addition of 1, 3,7-trimethyluric acid salts.

According to another embodiment, the total concentration of 1,3,7-trimethyluric acid salts in an aqueous solution provided hereby may be any amount between about 0.01% and about 20% (or more) by weight based on the total weight of the aqueous solution, including all percentages and ranges of percentages there between.

According to another embodiment, the total concentration of 1,3,7-trimethyluric acid salts in an aqueous solution provided hereby may be any amount between about 0.1% and about 20% by weight based on the total weight of the aqueous solution, including all percentages and ranges of percentages there between.

According to another embodiment, the total concentration of 1,3,7-trimethyluric acid salts in an aqueous solution provided hereby may be any amount between about 0.1% and about 25% by weight based on the total weight of the aqueous solution, including all percentages and ranges of percentages there between.

According to another embodiment, the total concentration of 1,3,7-trimethyluric acid salts in an aqueous solution provided hereby may be any amount between about 0.1% and about 30% by weight based on the total weight of the aqueous solution, including all percentages and ranges of percentages there between. In an alternate embodiment, 1,3,7-trimethyluric acid salts) may be added to a natural beverage in any amount provided that an aqueous solution or suspension results.

In addition, a composition according to the present disclosure also includes nutritional adjuvant materials including flavoring agents, colorants, viscosity modifiers, preservatives, chelating agents, antioxidants, surface modifiers and other nutritional adjuvant materials.

Other excipients include any substance which is generally recognized as promoting the health or function of a mammalian organism, including humans, or benefiting a composition useful thereof in terms of its efficacy, appearance, stability, consistency, aroma, or viscosity.

Such substances include, but are not limited to, other amino acids and their salts, vitamins, minerals, essential fatty acids, enzymes, mono-glycerides, di-glycerides, tri-glyceride ester oils (including, for example vegetable oils and animal fats) emulsifiers, hydrolyzed proteins, whey protein, stabilizers, flow modifiers, viscosity improvers, chelating agents, enzymes, surfactants, whether anionic, cationic or nonionic, and combinations thereof.

The total amount of the one or more nutritional adjuvant materials above present in a composition according to this disclosure is present in any amount between about 0.01% and about 50% by weight based on the total weight of said composition, including all percentages and ranges of percentages there between.

In addition to ingredients containing adjuvant materials, a composition according to one embodiment of the present disclosure also comprises one or more natural beverages.

A natural beverage, as used herein, is a beverage suitable for human or animal consumption which contains the pulp, juice or any other constituent of a naturally-occurring fruit, vegetable, or animal product whether from the wild, cultured, cultivated on a farm or otherwise domesticated by man.

Natural beverages include without limitation materials including, but not limited to as milk products, soy products, ice cream, yoghurt, citrus fruit juices, non-citrus fruit juices, and vegetable juices, or components of any of the foregoing, wherein said natural beverages are present in any effective amount to impart flavor to the compositions, which are any amount between about 0.1% and about 99% by weight based on the total weight of said composition, including all percentages and ranges of percentages there between.

In addition to ingredients containing adjuvant materials, a composition according to one embodiment of the present disclosure comprises one or more synthetic beverages. A synthetic beverage is any beverage that is not a natural beverage.

In general, a composition according to one embodiment of the present disclosure is provided by combining and mixing the ingredients selected, including 1,3,7-trimethyluric Sodium salt and any desired quantity of any one or more other ingredients specified herein.

One advantage of compositions according to this disclosure is that they may be packaged at pH levels as low as about pH 2, in the cold fill (room temperature).

Another advantage of compositions according to this disclosure is that the liquid or solid dry blends formulations can be easily be flavored without any additional taste maskers or taste modifiers to eliminate bitter taste.

Thus, it is evident that a composition according to one embodiment of the present disclosure is made palatable by a human subjects desiring to administer 1,3,7-trimethyluric acid orally in an aqueous mixture. Typical serving sizes may be any serving size in the range of about 1 milligram to about 50 grams, in an aqueous solution that is from about 5 ml to about 2500 ml in volume.

It's been surprisingly discovered the advantage of 9 position being an NH, offer two main advantages:

1) Possibility to synthesize water-soluble salts

2) no bitter aftertaste.

### Example 3: Formula Tablets or Capsules

1,3,7-trimethyluric acid salts—between 0.01% and about 20% (or more) by weight based on the total weight;

Lubricants: chosen from magnesium Stearate, stearic acid, L-Leucine, MCT powder, ascorbyl palmitate—between 0.1 and 10% alone or in any combination;

Binders: selected from maltodextrin, calcium carbonate, lactose, Micro crystalline Cellulose, Silicified microcrystalline cellulose, sugars of any type in amount of 0.1% and 99% by weight;

Anti-caking/flowing agents: Silicates such as precipitates silica, hydrophobic and hydrophilic silicates, calcium silicates but not limited to these in any combinations 0.01% to 10%

Production Method:

Tablets can be obtained by mixing the materials above in a suitable blender, the material is discharged and then tablets of any shape are made by direct compression to form tablets in a suitable tableting machine. The tablets can be obtained as for immediate release but also for delay release or controlled release as desired, using various available ingredients known in the arts to achieve such tablets; the tablets can also be obtained as flavored chewable, sublingual, fast dissolve or effervescent tablets using various systems of flavors and acids to achieve desire taste.

Capsules can be formed using any suitable encapsulation machine using all sizes available of capsule shells, gelatin, vegetarian capsules or DR capsules (delayed released).

### Example 4: Formulas for Soft Gels/Li-Caps

Suspend or dissolve the desired dose quantity of 1,3,7-trimethyluric acid salts in carrier chosen from: any suitable lipophilic agent or soybean oil, beewax, glycerine, MCT (medium chain tryglycerides) oil, coconut oil, CLA (conjugated linoleic acid), lecithin, fish oil, any suitable grade vegetable oil, hemp oil, animal origin oil but not limited to this; incorporate any desired dietary supplement and any desired amount of stimulants and form the softgel or licap using any suitable lab size or industrial size equipment.

### Example 5: Co-Crystals of 1,3,7-Trimethyluric Acid and its Salts or Derivatives, Theacrine, Liberine, Methyl-Liberine with Piceid

Co-crystals were prepared by a grinding method and solvent-based methods or vapor diffusion technique:

5.1. Equal amount of each of theacrine, libertine or methylliberine and piceid was added to a milling jar.

A suitable amount of solvent was added to the milled material; the solvent can be chosen from methanol, ethanol, acetonitrile, chloroform but not limited to these, and the material was ground for 20 minutes at 20 Hz;

5.2 for the solvent-based methods at ambient temperature, solid theacrine, libertine or methylliberine was added to a nearly saturated solution of piceid in ethanol and allowed to stir for about 24 hours before filtering. Single crystals were grown from a vapor diffusion experiment where a 1:1 mixture of piceid and theacrine, libertine or methylliberine was dissolved in a minimal amount of methanol vial. The vial was placed uncapped in a bigger vial containing water. The larger vial was capped, and after 2 days, rod-shaped crystals were harvested.

5.3 Single crystals of 1,3,7-trimethyluric acid and its salts or derivatives, theacrine, Liberine, methylliberine with piceid;

These co-crystals can be further used in formulations of dietary supplements or foods finished products by combining these with any dietary supplement ingredients in any amount; these formulation can be for oral administration in a form of ready to drink mixes, tables, capsules, softgels, gels or liquids;

Co-crystallization of 1,3,7-trimethyluric acid and its salts or derivatives, theacrine, Liberine, methylliberine with piceid is used here to improve bio-availability of such ingredients.

### Example 6: Bitterness Assessments Test

Masking bitterness and aftertaste of drink formulations is not an easy task and require the addition of masking or blockers bitterness chemicals components into the formulations. This process presents the disadvantage for formulators and could have a serious impact in overall cost.

Various bitterness assessments tests were conducted using triangle test method in order to compare formulations made with 1,3,7-trimethyluric acid or salt versus formulations made using theacrine (1,3,7,9-tetramethyluric acid);

The flavor profile on all formulations was kept in the same amounts in order to have no influence from it. The type of color and the color amount was the same in both formulas so that when dissolved into the water will match. A control sample was also prepared as “placebo” and given to both groups of panelist in a random order.

Two groups of three trained panelists were chosen for the triangle test in order to identify the odd samples.

To the first group of panelist, three drink samples were given in random order to taste: two of the same sample and one that was different:

First sample formula contains the following:

| Ingredient | Amount (mg) |
|---|---|
| 1,3,7-trimethyluric acid | 200 |
| Citric acid | 350 |
| Malic Acid | 200 |
| Fruit punch N&A Flavor | 450 |
| Sucralose | 100 |
| Red #40 | 5 |

The second formula contains the following:

| Ingredient | Amount (mg) |
|---|---|
| Theacrine(1,3,7,9- | 200 |
| tetramethyluric acid) | |
| Citric acid | 350 |
| Malic Acid | 200 |
| Fruit punch N&A Flavor | 450 |
| Sucralose | 100 |
| Red #40 | 5 |

All samples formulas were dissolved separately into 10 Oz of cold water and shook to dissolve. The cups were filled in with each formula drink and were given to the first group of panelists in random order. Panelists were then asked to evaluate the overall attributes and identify the odd sample

The same kind of experiment was repeated using the second group of panelists comparing now the 1,3,7-trimethyluric sodium salt versus theacrine (1,3,7,9-tetramethyluric acid) with the addition of caffeine anhydrous in each formulation on equal amounts.

First formula contains the following:

| Ingredient | Amount (mg) |
|---|---|
| 1,3,7-trimethyluric sodium salt | 200 |
| Caffeine anhydrous | 200 |
| Citric acid | 350 |
| Malic Acid | 200 |
| Fruit punch N&A Flavor | 450 |
| Sucralose | 100 |
| Red #40 | 5 |

Second formula contains the following:

| Ingredient | Amount (mg) |
|---|---|
| Theacrine (1,3,7,9- | 200 |
| tetramethyluric acid) | |
| Caffeine anhydrous | 200 |
| Citric acid | 350 |
| Malic Acid | 200 |
| Fruit punch N&A Flavor | 450 |
| Sucralose | 100 |
| Red #40 | 5 |

Caffeine was added in both formulations in equal amounts and the flavor profiles were kept the same as in the first set of experiments. The first sample and the second sample formulas were dissolved separately into 10 Oz of cold water and shook to dissolve. 3 cups, 2 of which were filled in with first formula drink and a third cup filled with the second sample formula drink, were given to the first group of panelists in random order. Panelists were then asked to evaluate the overall attributes and identify the odd sample.

In this study, the panelists detected a significant difference between formulations. The bitterness and aftertaste was unanimously identified by all panelists to be significant and intolerable on the samples formulated with theacrine (1,3,7,9-tetramethyluric acid) and almost no bitterness or aftertastes could be noticed by any panelist when tasting formulations where the 1,3,7-trimethyluric acid or its sodium salt was used. No significant difference was noticed between the flavor and taste of control (placebo) sample and the formulations made with 1,3,7-trimethyluric acid alone or when combined with caffeine.

It was surprisingly concluded that the methylated position 9 on the theacrine molecule structure directly influences the bitterness and aftertaste in drinks formulations. Therefore, the advantages of using 1,3,7-trimethyluric acid or its sodium salt were obvious.

There should be understood that the above examples of co-crystals are just examples and are not a limitation present disclosure to any other co-crystallizations processes and any other combinations of such ingredients and the way they can be used to formulate dietary supplements or foods.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A composition comprising one or more compounds selected from the group of:
a) soluble 1,3,7-trimethyluric acid salts;
b) co-crystals of 1,3,7-trimethyluric acid;
c) 1,7-dimethyluric acid salts 1-methyluric acid salts, 3,7-dimethyluric acid salts, and/or 9-methyluric acid salts, or
d) co-crystals of 1,7-dimethyluric acid, 1-methyluric acid, 3,7-dimethyluric acid, 9-methyluric acid and/or their salts.

2. The composition of claim 1, wherein the soluble 1,3,7-trimethyluric acid salts are present in an amount between about 0.01% and about 10% by weight based on a total weight of the composition.

3. The composition of claim 1, wherein the soluble 1,3,7-trimethyluric acid salts, the 1,7-dimethyluric acid salts, the 1-methyluric acid salts, and/or the 3,7-dimethyluric acid salts are present in an amount between about 0.01% and about 10% by weight based on a total weight of the composition.

4. The composition of claim 1, wherein the composition further comprises one or more materials selected from the group of purines, xanthines or methylated xanthines, methylated forms of uric acid, synephrine, methyl synephrine, citrus aurantium, yerba mate, green tea extracts, catechols, yohimbine species, cocoa extract, coffee bean extract, chlorogenic acid, raspberry ketone, hydroxycitrate, conjugated linoleic acid, naringin, black pepper, guggul, guarana, cola nut, beta-phenylethylamine, colostrum, coleus forskohlli, rhodiola, garcinia cambogia, ashwagandha, ginseng species, gingko, scutellaria, liquorice, dehydroepiandrosterone (DHEA), pregnenolone, tyrosine, N-Acetyl Tyrosine, glucuronolactone, taurine, choline, CDP-choline, alpha glycerol phosphatidyl choline, L-carnitine and L-carnitine derivatives, 5-hydroxytryptophan, melatonin, γ-aminobutyric acid), beta hydrohybutyric acid salts, phenylethylamine, scetetium tortosum, Dendrobium candidum, quinoline quinone, ubiquinone, nicotinamide riboside, Huperzine A, L-dopa, Pinus sylvestris, 2-(dimethylamino)ethanol, medium chain triglyceride, pterostilbenes, steroids, resveratrol, berberine, D-pinitol, tyramine, hordenine, agmatine, betaine, alanine, beta alanine, branch chain amino acids, or gamma-butyro betaine in an amount between about 0.01% and about 10% by weight based on a total weight of the composition.

5. The composition of claim 4, wherein the composition further comprises one or more excipients selected from the group of flavoring agents, colorants, viscosity modifiers, preservatives, fragrances, amino acids and their salts, vitamins, minerals, essential fatty acids, enzymes, co-enzymes, mono-glycerides, di-glycerides, tri-glyceride ester oils emulsifiers, hydrolyzed proteins, whey protein, stabilizers, flow modifiers, viscosity improvers, chelating agents, anti-oxidants, anti-microbials benzoates or sorbates, alcohols, esters of para-hydroxybenzoic acid, propionates, preservatives, or surfactants, including anionic, cationic and/or nonionic surfactants.

6. The composition of claim 5, wherein the one or more excipients is present in an amount between about 0.01% and about 10% by weight based on a total weight of the composition.

7. The composition of claim 6, wherein the composition is included in a beverage selected from the group of energy drinks, vitamins and mineral water, alkaline or buffered water, proteins and milk products, soy products, ice cream, yoghurt, fruit juices, vegetable juices, or combinations thereof.

8. The composition of claim 7 wherein the beverage includes one or more natural components, and wherein the one or more natural components are present in an amount of from about 0.1% to about 99% by weight based on the total weight of the composition.

9. The composition of claim 8, wherein the composition further comprises an anti-microbial preservative.

10. The composition of claim 9, wherein the anti-microbial preservative comprises a preservative selected from the group of one or more esters of para-hydroxy benzoic acid, propionates, one or more sorbates, or combinations thereof.

11. The composition of claim 10, wherein the composition further includes a buffering solution selected from the group of: phosphates, phosphoric acid, acetates, carbonates or bicarbonates, citrates, or combinations thereof.

12. A dietary supplement or food product, wherein the dietary supplement or food product comprises the composition of claim 1.

13. A method for administering a composition to a mammalian subject, the method comprising the steps of: preparing the composition of claim 10; and orally administering the composition to the mammalian subject.

14. (canceled)

15. (canceled)

16. The dietary supplement or food product of claim 12, wherein the dietary supplement or food product is in a dosage form, and wherein the dosage form is solid, liquid, or semi-solid.

17. The dietary supplement or food product of claim 12, wherein the dietary supplement or food product is in a dosage form, and wherein the dosage form is solid or aqueous.

18. The dietary supplement or food product of claim 12, wherein the dietary supplement or food product is in a dosage form, and wherein the dosage form is aqueous, topical cream, gel, or ointment.

19. The dietary supplement or food product of claim 18, wherein the composition further comprises at least one acid regulator or buffer having a pKa of from about 1.5 to about 9.0.

20. The composition of claim 1, wherein the soluble 1,3,7-trimethyluric acid salts is 1,3,7-trimethyluric sodium salt.
